(19) United States
(12) Patent Application Publication
(10) Pub. No.: US 2020/0329781 A1
(43) Pub. Date: Oct. 22, 2020

(54) STRING WEAR-SUPPORTED SKIN GUARD

(71) Applicant: KING SAUD UNIVERSITY, RIYADH (SA)

(72) Inventor: MOHD AFTAB ALAM, RIYADH (SA)

(21) Appl. No.: 16/390,176

(22) Filed: Apr. 22, 2019

(51) Int. Cl. A41B 9/00; A61F 13/00; A61F 13/68

(52) U.S. Cl. CPC A41B 9/002; A61F 2013/15024; A61F 13/68; A61F 13/00063

(57) ABSTRACT

The string wear-supported skin guard includes a waist strap, first and second spaced thigh strings that depend from the waist strap, and one or more detachable absorbent components connected to at least one of the thigh strings. Opposing ends of each thigh string are connected to the waist strap, such that a generally U-shaped loop is defined by each thigh string. Each thigh string can include a generally C-shaped hip string. When the string wear-supported skin guard is worn by a user, the waist strap encircles the user's waist, the hip strings encircle the user's hips, and the thigh strings extend between the hip region, inner thigh area, and buttocks of the user. The one or more absorbent components overly the inner thigh of the user to provide a skin guard for the prevention and treatment of skin irritation and skin rashes in the inner thigh area.

### BACKGROUND

### 1. Field

The disclosure of the present patent application relates to skin protection, and particularly to a string wear-supported skin guard for the prevention and treatment of skin irritation and skin rashes in the inner thigh area.

### 2. Description of the Related Art

Skin irritation and skin rashes can be developed in the inner thigh area due to accumulation of sweat and other bodily fluids, contact or friction between the skin of the inner thighs and the scrotum, and contact or friction between the inner thigh and the hem of some undergarments. In severe cases, the skin irritation or rash can hamper movement or physical activity. Further, the untreated skin irritation and skin rashes may also lead to microbial infections, for example bacterial or fungal infections.

Thus, a string wear-supported skin guard for the prevention and treatment of skin irritation and skin rashes in the inner thigh area and solving the aforementioned problems is desired.

### SUMMARY

A string wear-supported skin guard includes a waist strap, strings, and at least one detachable absorbent component. The thigh strings are attached to the waist strap. The first and second spaced thigh strings depend from the waist strap. The at least one detachable absorbent component is connected to at least one of the thigh strings. Opposing ends of each thigh string can be connected to the waist strap, such that a generally U-shaped loop is defined by each thigh string. Each thigh string can include a respective C-shaped (or semicircular) hip string. The string wear-supported skin guard can be worn such that the waist strap encircles a user's waist, each of the hip strings encircle a respective hip (upper part) of the user, and each thigh string extends between a respective inner thigh area, hip region, and buttocks of the user. The absorbent components are configured for overlying the inner thigh of the user to provide a skin guard for the prevention and treatment of skin irritation and skin rashes in the inner thigh area.

These and other features of the present disclosure will become readily apparent upon further review of the following specification and drawings.

### BRIEF DESCRIPTION OF THE DRAWINGS

FIG. 1 is a front view of a first embodiment of string wear-supported skin guard according to the present teachings.

FIG. 2 is an environmental, front view of the first embodiment of the string wear-supported skin guard.

FIG. 3 is an environmental, front view of the string wear-supported skin guard, showing an alternative embodiment of the detachable absorbent component.

FIG. 4 is a top view of the detachable absorbent component configured as a flat sheet.

FIG. 5 is a perspective view of the detachable absorbent component configured as a tube.

FIG. 6 is a front view of an alternative embodiment of a string wear-supported skin guard according to the present teachings.

FIG. 7 is a front, environmental view of the string wear-supported skin guard shown in FIG. 6.

FIG. 8 is a top view of the detachable absorbent component of the skin guard shown in FIG. 7.

FIG. 9 depicts exemplary hook-and-loop fasteners that can be used in the embodiments described herein.

FIG. 10 depicts exemplary snap fasteners that can be used in the embodiments described herein.

FIG. 11a depicts a rectangular cross-section of an exemplary string suitable for use in the embodiments described herein.

FIG. 11b depicts a circular cross-section of an exemplary string suitable for use in the embodiments described herein.

FIG. 11c depicts a triangular cross-section of an exemplary string suitable for use in the embodiments described herein.

FIG. 11d depicts a square cross-section of an exemplary string suitable for use in the embodiments described herein.

FIG. 11e depicts a pentagonal cross-section of an exemplary string suitable for use in the embodiments described herein.

FIG. 11f depicts a hexagonal cross-section of an exemplary string suitable for use in the embodiments described herein.

Similar reference characters denote corresponding features consistently throughout the attached drawings.

### DETAILED DESCRIPTION OF THE PREFERRED EMBODIMENTS

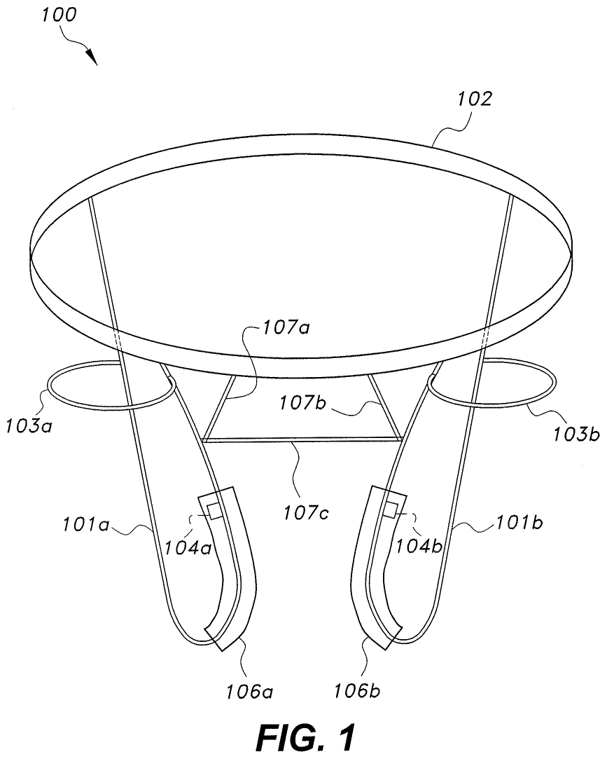

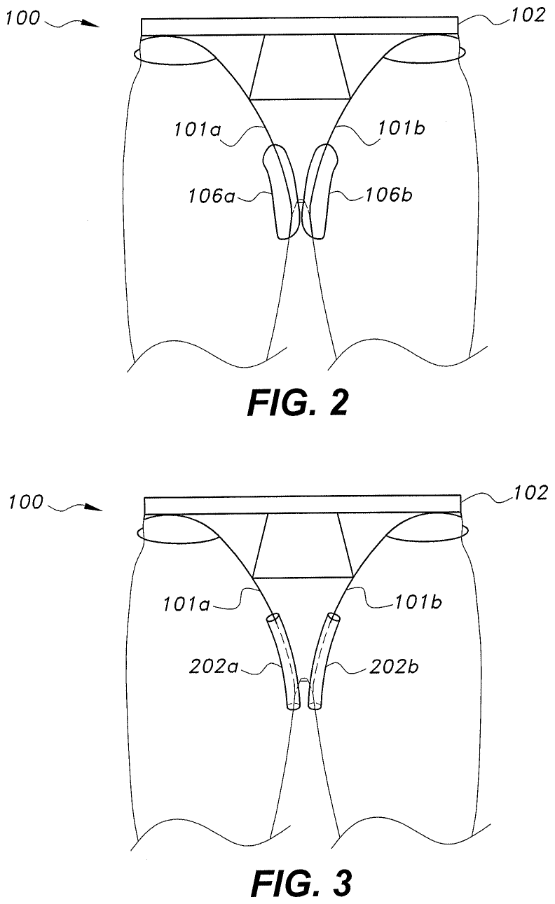

FIGS. 1-3 show a first embodiment 100 of the string wear-supported skin guard, generally designated as 100. The string wear-supported skin guard 100 includes a waist strap 102, first and second spaced thigh strings 101a and 101b that depend from the waist strap 102, and the detachable absorbent components 106a, 106b, connected to the respective thigh strings 101a, 101b. Opposing ends of each thigh string 101a, 101b are connected to the waist strap, such that a generally U-shaped loop is defined by each thigh string 101a, 101b. Each thigh string 101a, 101b can include a respective hip string 103a, 103b. Opposing ends of each hip string 103a, 103b can be attached to the respective thigh string such that a generally C-shaped (or semicircular) loop is formed by each hip string 103a, 103b. The string wear-supported skin guard 100 may further include optional stabilizing strings. For example, in an embodiment, the string wear-supported skin guard 100 may include a stabilizer string 107c that connects the thigh strings 101a and 101b. In another embodiment, three stabilizer strings 107a, 107b and 107c can be arranged as generally U-shaped. The upper ends of stabilizer strings 107a and 107b are connected to the waist strap and the lower ends of stabilizer strings 107a, 107b are attached to the respective thigh strings 101a, 101b. The stabilizer string 107c connects the thigh strings 101a, 101b at the lower ends of stabilizer strings 107a and 107b. The strings 101a, 101b may further include fasteners 104a, 104b, respectively, for releasably attaching to mating fasteners on the absorbent component, as described in detail below. The string wear-supported skin guard can be worn such that the waist strap 102 encircles a user's waist, each of the hip strings encircle a respective hip of the user, and the thigh strings extend between the inner thigh area, hip region and buttocks of the user as shown in FIGS. 2 and 3. The detachable absorbent component is configured for overlying the inner thigh of the user to provide a skin guard for the prevention and treatment of skin irritation and skin rashes in the inner thigh area. The thigh string 101a, 101b pass over the detachable absorbent components placed in the inner thigh area, while the detachable absorbent components remain in contact with the skin of the inner thigh area.

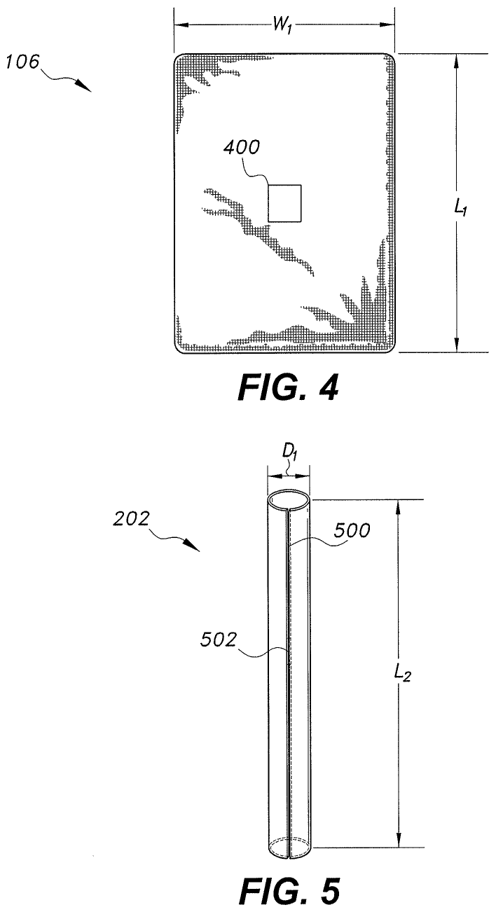

The detachable absorbent component can include a disposable, absorbent paper or fabric material. As shown in FIGS. 2 and 4, detachable absorbent component can be in the form of absorbent sheets 106a and 106b that are releasably attachable to thigh strings 101a and 101b, respectively. The sheets 106a, 106b are configured for overlying the inner thigh of the user to protect the user's skin. In the embodiments shown in FIGS. 1, 2, and 4, sheets 106a and 106b are flexible and flat or planar pieces of material. A length L₁ and the width W₁ of the sheets 106, can be configured to fit the particular user. The sheets 106a, 106b can include mating fasteners 400 for releasably attaching to fasteners 104a and 104b, respectively. The one or more fasteners can be attached at different levels of the thigh strings 101a, 101b and at different locations on the detachable absorbent component, as needed.

Alternatively, the detachable absorbent component can be configured as absorbent tubes 202a, 202b, as shown in FIGS. 3 and 5. Each thigh string 101a, 101b extends through a slot 500 of a respective tube 202a, 202b. Releasable fasteners 502 can be included on an inner wall of each tube 202a and 202b for securing tubes 202a, 202b to the fasteners 104a, 104b of strings 101a, 101b, respectively. The tubes 202a, 202b have a diameter D₁ and length L₂, configured to fit in the inner thigh area of the particular user.

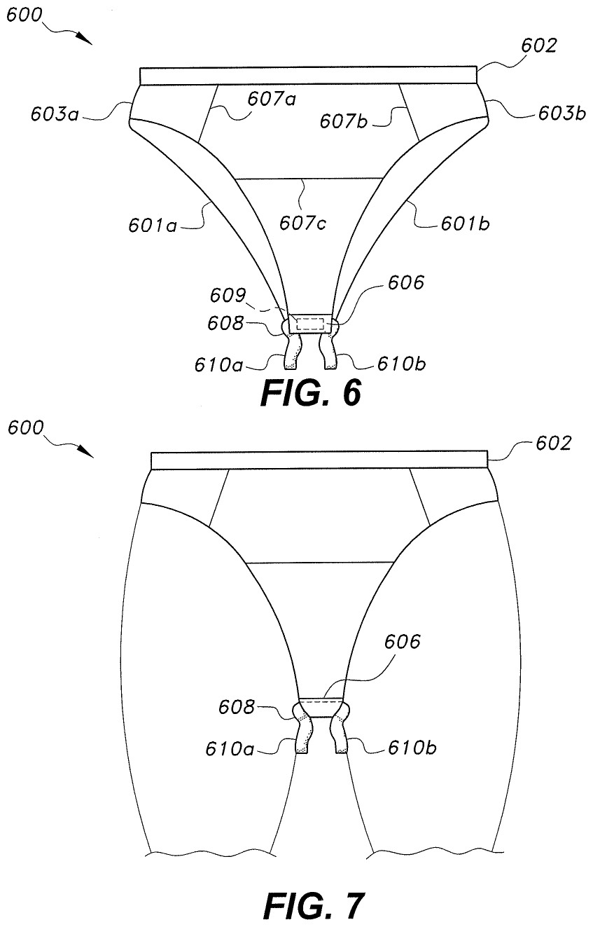

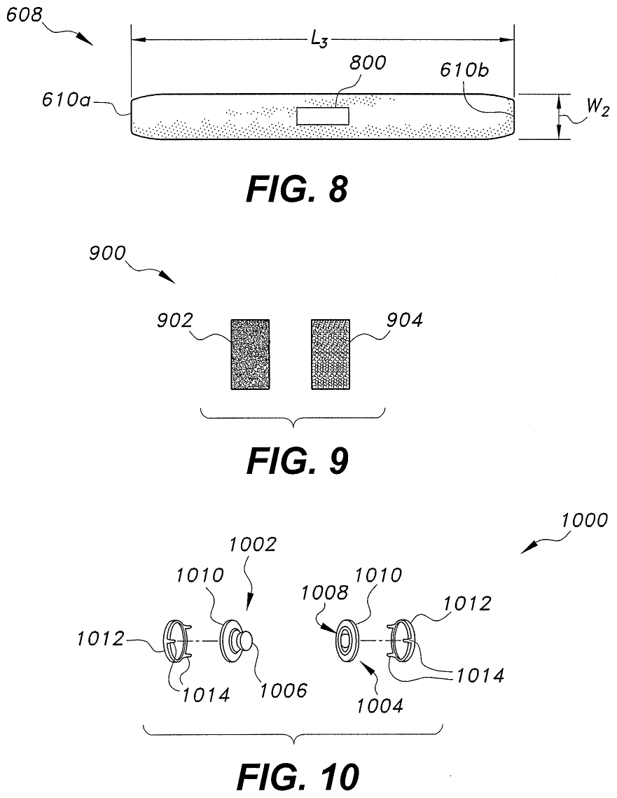

FIG. 6 illustrates another embodiment of the string wear-supported skin guard, generally designated as 600. The string wear-supported skin guard 600 can have a waist strap 602, thigh strings 601a and 601b, and stabilizer strings 607a, 607b and 607c. The stabilizer string 607a connects the front portion of the thigh string 601a with waist strap 602, and the stabilizer string 607b connects the front portion of the thigh string 601b with waist strap 602. The stabilizer string 607c extends horizontally and connects the front portion of the thigh string 601a with the front portion of the thigh string 601b. The skin guard 600 is generally similar to skin guard 100. Unlike skin guard 100, however, the skin guard 600 includes a generally rectangular base strap 606. One side of the base strap 606 is attached to the first thigh string 601a and an opposing side of the base strap is attached to the second thigh string 601b. The base strap 606 can include a fastener 609 for detachably receiving a detachable absorbent component 608 thereon. The detachable absorbent component 608 can be a single sheet having foldable sides 610a and 610b and a releasable fastener 800 as shown in FIGS. 7 and 8. The sheet 608 is configured for protecting the inner thighs as well as the perineum of the user. Further, unlike thigh strings 101a, 101b, thigh strings 601a, 601b are generally circular and are attached to the waist strap 602 by linear connector strings 603a and 603b, respectively.

The releasable fasteners described herein can include hook-and-loop fasteners, snap fasteners, or other appropriate releasable fasteners known in the art. As shown in FIG. 9, the releasable fasteners can include a hook and loop fastener 900, including a first patch 902 of hook fastener and a second patch 904 of mating loop fastener. In an alternative embodiment, the releasable fasteners can include mating snap fasteners 1000, including a first male snap fastener 1002 and a second mating female snap fastener 1004. The male snap fasteners 1002 include a protrusion 1006 with an enlarged head that snaps into an orifice 1008 of the female snap fasteners 1004. Both the male snap fasteners 1002 and the female snap fasteners 1004 include a disc-shaped body 1010. The male snap fasteners 1002 and the female snap fasteners 1004 can be attached to a desired structure using ring-shaped connectors 1012. The ring-shaped connectors 1012 include foldable tabs 1014 for cinching the snap fasteners to the desired structure.

It should be understood that the term “string,” as used herein, can refer to threads of cotton or other suitable material that are twisted together to form a thin cord having a small cross-section. Furthermore, the term “string” also includes elastic or non-elastic materials woven or knitted together to form a flat, round or angular cord of suitable dimension. The resilient rubber strings of suitable dimension are also included under the definition of “string.” It should further be understood that the term “strap” refers to a sheet of material having a greater cross-section or width than a cross-section or width of the “string.” For example, the “waist strap” can have a greater width than the width of the string and is elastic in nature. In an embodiment, the “waist strap” can be formed from elastic and non-elastic materials. Similarly, the “base strap” can have a greater width than the width of the string. In an embodiment, the “base strap” can be formed from a sheet of woven or non-woven material.

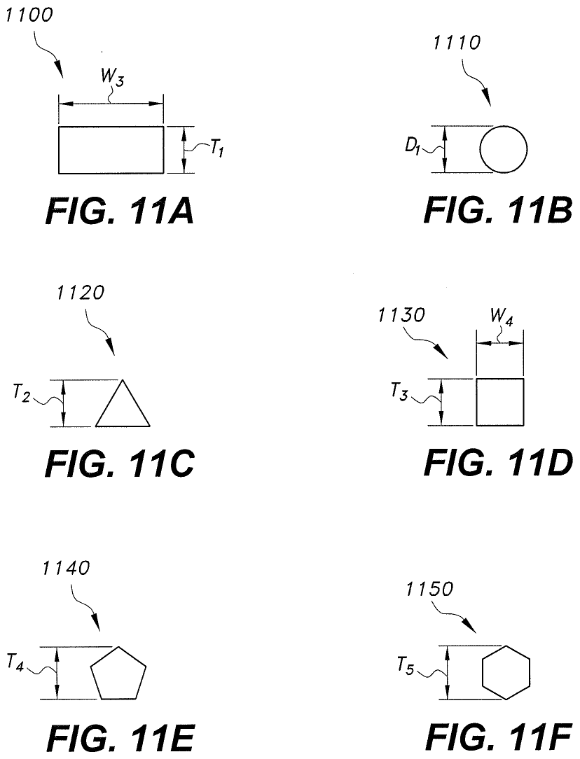

FIGS. 11a through 11f illustrate strings of various cross-sections suitable for use with any of the embodiments of the string wear-supported skin guard. FIG. 11a shows a flat string 1100 with a rectangular cross-section having a width of W₃ and a thickness of T₁. Preferably, W₃ is from 3 mm to 12 mm, more preferably from 4 mm to 10 mm and most preferably from 5 mm to 8 mm. Preferably, T₁ is from 0.5 mm to 2 mm and most preferably from 0.5 mm to 1 mm. FIG. 11b shows a string 1110 with a circular cross-section having a diameter or thickness of D₁. Preferably, D₁ is from 2 mm to 5 mm. FIG. 11c through 11 f show strings with polygonal cross sections that are all suitable for use with any of the embodiments described herein. FIG. 11c shows a string 1120 that has a triangular cross-section having a thickness of T₂. FIG. 11d shows a string 1130 that has a square cross-section having a thickness of T₃ and width of W₄, wherein T₃=W₄. FIG. 11e shows a string 1140 that has a pentagonal cross-section having a thickness of T₄. FIG. 11f shows a string 1150 that has a hexagonal cross-section having a thickness of T₅. Preferably, T₂, T₃, T₄ and T₅ are from 2 mm to 5 mm in thickness.

The detachable absorbent component is soft, flexible, non-adhesive, and may be made from paper, cotton, linen textile, or other suitable material. In some embodiments, the detachable absorbent component is drug-free. In other embodiments, the detachable absorbent component includes at least one drug substance for the treatment of skin irritation and skin rashes. The at least one drug substance can include an antimicrobial agent selected from the group consisting of nystatin, clotrimazole, miconazole, griseofulvin, amphotericin B, econazole, ketoconazole, oxiconizole, sulconazole, naftifine, terbinafine, butenafine HCl, ciclopirox, haloprogin, tolnaftate and thymol. Suitable pharmaceutical excipients can be used with the antimicrobial agent. The pharmaceutical excipients can be selected from talc, calcium silicate, magnesium silicate, colloidal silicon dioxide, citric acid anhydrous, citric acid monohydrate, fumaric acid, benzalkonium chloride, benzethonium chloride, benzoic acid, potassium benzoate, potassium sorbate, propylparaben, propylparaben sodium, sodium benzoate, ethycellulose, hydroxypropyl cellulose, hydroxypropyl methylcelluloce, hydroxypropyl methylcelluloce phthalate, hypromellose acetate succinate, hypromellose phthalate, maltodextrin, methacrylic acid copolymer, methacrylic acid copolymer dispersion, methylcellulose, polyvinyl acetate phthalate, titanium dioxide and sodium lauryl sulfate. The antimicrobial agent may be absorbed within or coated on a surface of the detachable absorbent components.

It is to be understood that the string wear-supported skin guard for the prevention and treatment of skin irritation and skin rashes in the inner thigh area is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A string wearer-supported skin guard, comprising:
a circular waist strap;
first and second thigh strings connected to the circular waist strap at opposing ends thereof, wherein each of the first and second thigh strings are non-manually, self-length adjustable, unitary generally U-shaped strings;
a pair of non-manually, self-length adjustable, unitary C-shaped hip strings, wherein each of the non-manually, self-length adjustable, unitary generally C-shaped hip strings are attached to a respective first and second thigh string;
a stabilizing string attached to the first and second thigh strings, wherein the stabilizing string is generally U-shaped, having opposing ends attached to the circular waist strap and opposing corners attached to the first and second thigh strings; and
At least one detachable absorbent component connected to at least one of the first and second thigh strings, the at least one of the first and second thigh strings passing over the at least one detachable absorbent component.

2-5. (canceled)

6. The string wearer-supported skin guard according to claim 1, wherein each of the first and second thigh strings includes a first detachable fastener and the at least one detachable absorbent component includes a second detachable fastener for Mattingly engaging the first detachable fastener of a respective one of the first and second thigh strings.

7. the string wearer-supported skin guard according to claim 1, wherein the at least one detachable absorbent component includes an absorbent paper material.

8. the string wearer-supported skin guard according to claim 1, wherein the at least one detachable absorbent component includes an absorbent fabric material.

9. the string wearer-supported skin guard according to claim 1, wherein the at least one detachable absorbent component includes a flat sheet of absorbent material.

10. The string wearer-supported skin guard according to claim 1, wherein the at least one detachable absorbent component includes a tubular absorbent material.

11. The string wearer-supported skin guard according to claim 1, wherein the at least one detachable absorbent component includes at least one antimicrobial agent.

12-14. (canceled)
